(19) United States
(12) Patent Application Publication
(10) Pub. No.: US 2016/0199212 A1
(43) Pub. Date: Jul. 14, 2016

(54) BRACE FOR IMMOBILISING A TRAUMATIZED OR OPERATED SHOULDER

(71) Applicant: ORTHOSERVICE AG, Chiasso (CH)

(72) Inventors: Francesco TURCONI, Monza (MB) (IT); Paolo ROSSI, Chiasso (CH)

(73) Assignee: ORTHOSERVICE AG, Chiasso (CH)

(21) Appl. No.: 14/912,214

(22) PCT Filed: Sep. 9, 2014
(86) PCT No.: PCT/IB2014/064340

§ 371 Date: Feb. 16, 2016

(30) Foreign Application Priority Data
Sep. 10, 2013 (IT) MI2013A 001491

(51) Int. Cl. A61F 5/37

(52) U.S. Cl. CPC A61F 5/3753; A61F 5/3738

(57) ABSTRACT

A brace for immobilising a traumatized or operated shoulder including: at least one cushion configured to at least partly adhere to the body of a user having a traumatized or operated shoulder to be immobilised; at least one arm support sling extends from the cushion and with it delimits a compartment for housing a forearm of the user corresponding to the traumatized or operated shoulder; at least one abdominal band configured to envelope and/or surround at least one abdominal and lumbar area of the user; at least one support harness engaged to the cushion to support the cushion at a predetermined position and level. The support harness includes at least one rest portion configured to engage at least one shoulder of the user to support the cushion and the arm support sling. The support harness is configured to engage, resting thereon, the user's traumatized or operated shoulder.

The present invention refers to a brace for immobilising a traumatized or operated shoulder.

The present invention is suitable for being used in the orthopaedic medical field and, in particular, it refers to the field of braces and/or di similar support structures suitable for keeping operated or traumatised body parts in the correct position.

In detail, the object of the present invention focuses on braces intended for immobilizing shoulders of patients who have undergone or have been subjected to: repair of rotator cuffs; subluxations; dislocations; Bankart procedures; repair of soft tissues following traumas; external rotation immobilisation; plastic capsular repair procedures or anterior/posterior stabilisations procedures.

As known, braces intended for immobilising traumatized or operated shoulders are usually equipped with a cushion that is placed between the chest and the limb of the patient corresponding to the shoulder involved. The cushion is generally provided with an arm support sling configured to receive the forearm of the respective user. The cushion usually has a series of belts engaged with it that allow the brace to be attached to the body of the user so that the forearm, inserted into the arm support sling and, consequently, the corresponding traumatized or operated shoulder, remain locked in a predetermined position.

Generally, the aforementioned braces are provided with an abdominal band configured to envelop and surround the abdominal/lumbar regions of the user to keep the cushion adherent to the body of the same.

Such braces are also provided with a support belt the ends of which engage the cushion from above so as to form a strap the top of which is configured to rest on the contralateral shoulder of the user, i.e. the healthy shoulder not involved in the trauma or operation.

When worn, the belt or support strap of the brace rests on the shoulder opposite the traumatized or operated shoulder and the arm inserted in the arm support sling engaged to the cushion, extending diagonally to the sagittal plane of the user.

The belt or support strap is therefore placed under tension by the arm of the user that tends to pull the arm support sling and the cushion downwards, by gravity. Consequently, the belt or support strap, also pulled downwards by the relaxed arm, exerts a continuous pressure, in an oblique direction, on the base of the user's neck.

Although known braces using a cushion for immobilizing traumatised shoulders allow satisfactory rehabilitation thereof, the Applicant has found that they are not without drawbacks and they can be improved in various aspects, mainly in relation to comfort during use, the bodily well-being of the user, the correct position of the traumatized or operated shoulder, the degree of immobilization thereof, as well as the speed and times needed to rehabilitate the traumatised parts and allow the correct recovery of the kinetic chains involved.

In particular, the Applicant has found that all braces using a cushion for immobilizing traumatised shoulders are provided with belts or support straps that work on the contralateral shoulder generating a series of undesired problems.

Firstly, the diagonal orientation of the belts or support straps, typical of braces using a cushion for immobilizing traumatised shoulders, determines a continuous pressure on the base of the user's neck that, in the long term, begins to cause soreness or even acute pain that can lead to neck pain and/or similar pathologies. In detail, contralateral straps and belts work directly and transversally on the neck/shoulder joint relative to the healthy shoulder significantly stressing both the muscles and the nerve endings in this area. The sensations of discomfort, soreness and pain are rendered more acute and aggravated by the fact that such braces must be worn for particularly long periods, of the order of weeks.

Consequently, in order to compensate for unpleasant sensations, as well as the pains that are generated during the use of the aforementioned braces, users can take up incorrect positions and/or postures that lead to further drawbacks. Soreness and pain can even lead to the voluntary removal of the brace with serious consequences for the traumatized or operated shoulder.

Among the various incorrect postures and/or positions that users tend to take up during the use of such braces there is the lifting of the traumatized or operated shoulder, with consequent rotation of the scapula: this generates incorrect positioning with respect to the desired immobilization and complicates the rehabilitation procedures and the times needed for them.

The main purpose of the present invention is to provide a brace for immobilising a traumatized or operated shoulder capable of solving the problems encountered in the prior art.

A purpose of the present invention is to provide a brace for immobilising a traumatized or operated shoulder that is comfortable and does not generate sensations of soreness and/or even physical pain.

A further purpose of the present invention is to propose a brace for immobilising a traumatized or operated shoulder that ensures that the correct body position and posture are maintained.

A purpose of the present invention is to provide a brace for immobilising a traumatized or operated shoulder that is capable of simplifying and shortening, in terms of time, the rehabilitation procedures.

The purposes specified above, and yet others, are substantially accomplished by a brace for immobilising a traumatized or operated shoulder as expressed and described in the following claims.

As an example, it will be given the description of a preferred, but not exclusive, embodiment of a brace for immobilising a traumatized or operated shoulder in accordance with the present invention.

The description will be made hereafter with reference to the attached drawings, provided for indicating and therefore not limiting purposes, in which:

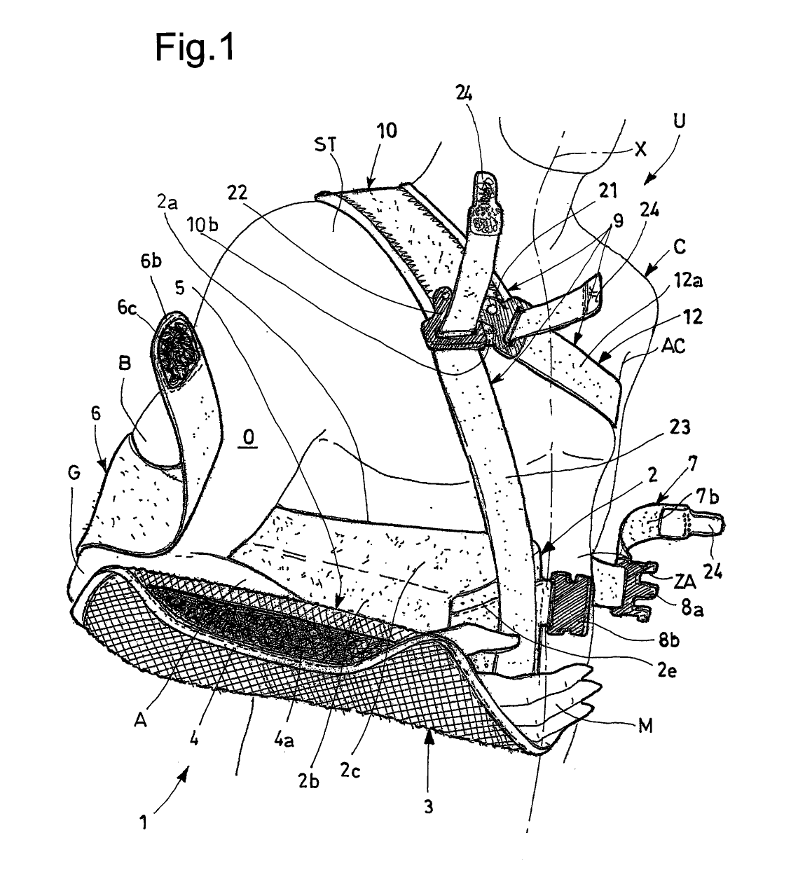

FIG. 1 is a schematic representation in frontal perspective view of a brace for immobilising a traumatized or operated shoulder of a user, in accordance with the present invention, shown while it is worn and adjusted;

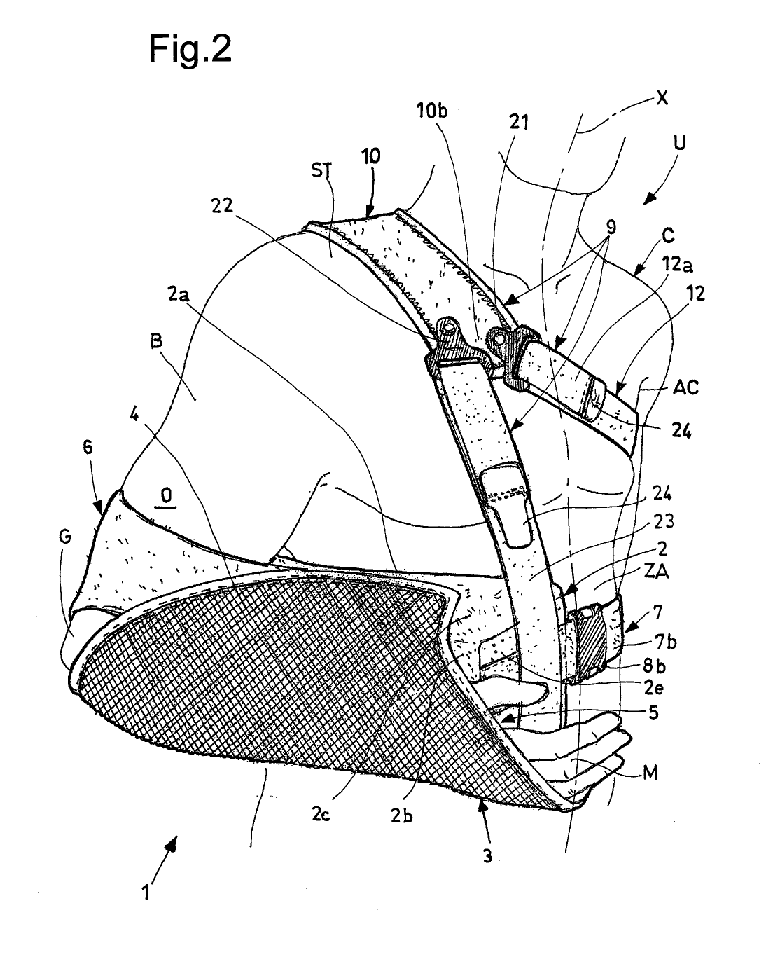

FIG. 2 is a further schematic representation in frontal perspective view of the brace according to FIG. 1 represented in worn condition;

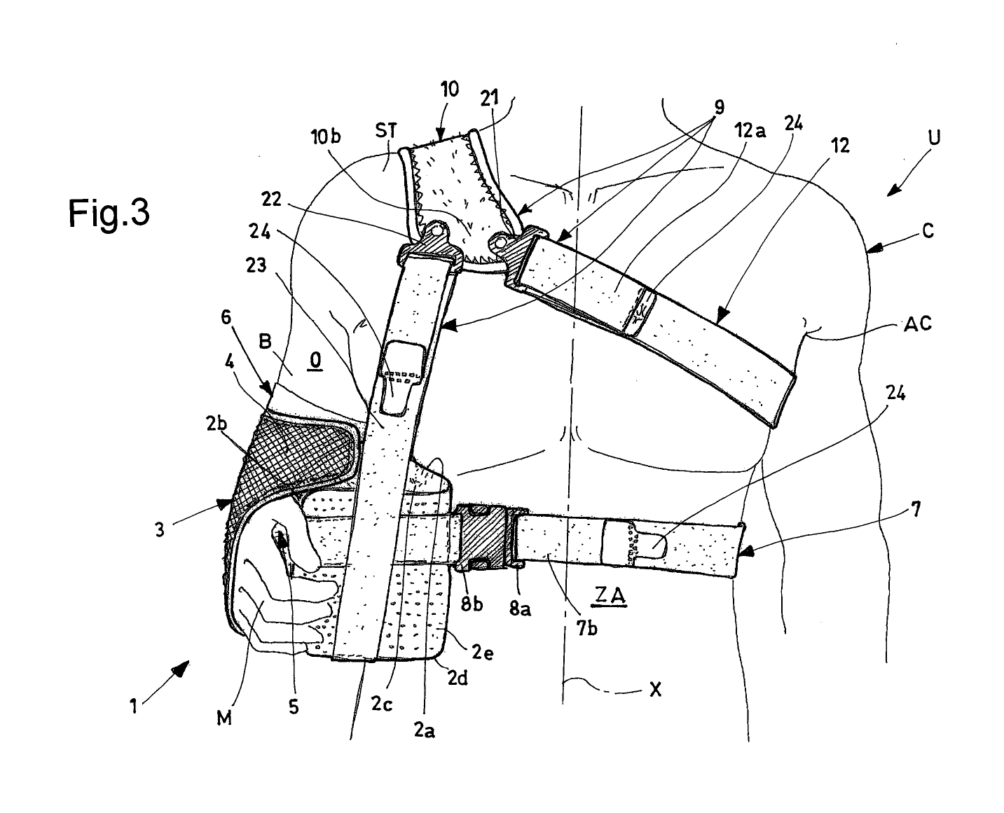

FIG. 3 is a schematic representation in frontal view of the brace according to FIGS. 1 and 2 shown in worn condition;

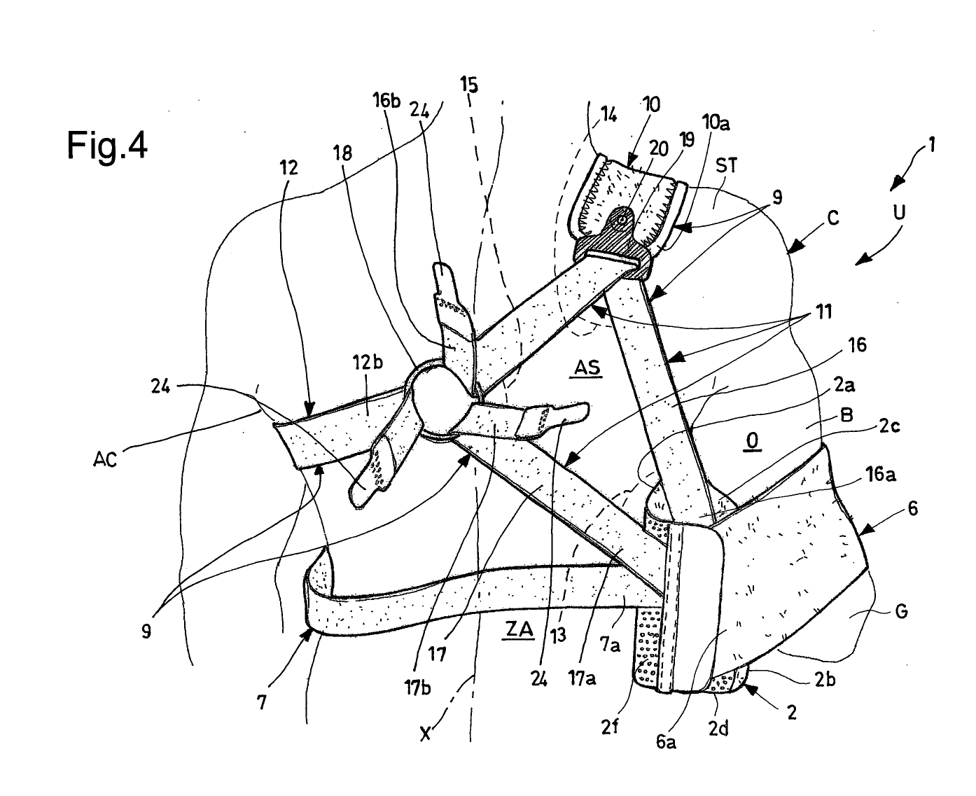

FIG. 4 is a schematic representation in rear view of the brace according to FIGS. 1 to 3 represented while it is worn and adjusted;

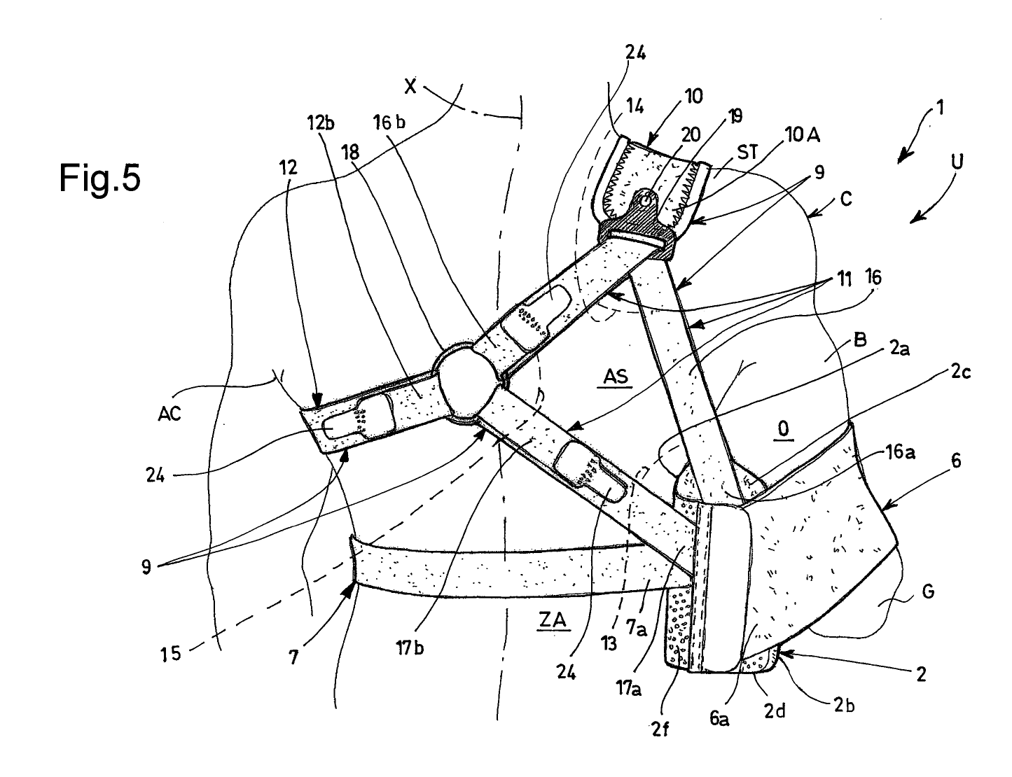

FIG. 5 is a schematic representation in rear view of the brace according to FIGS. 1 to 4 shown in worn condition.

With reference to the attached figures, reference numeral 1 wholly indicates a brace for immobilising a traumatized or operated shoulder, in particular for shoulders subjected to the repair of rotator cuffs, subluxations, dislocations, Bankart procedures, repair of soft tissues following traumas, external rotation immobilisation, plastic capsular repair or anterior/posterior stabilisations procedures, in accordance with the present invention.

As can be seen in the attached figures, the brace 1 comprises at least one cushion 2 suitable for at least partly adhering to the body C of a user U having a traumatized or operated shoulder ST to be immobilised.

In detail, the cushion 2 has a substantially elongated configuration on which it is possible to identify at least one inner surface 2a (FIGS. 1 to 5) configured to remain resting against the body C of the user U and at least one outer surface 2b (FIGS. 1 to 5) facing the opposite side with respect to the inner surface 2a, suitable for receiving, in contact the forearm A (FIG. 1) of the user U corresponding to the traumatized or operated shoulder ST.

The cushion 2 also has at least one upper surface 2c (FIGS. 1 to 5) and at least one lower surface 2d (FIGS. 3 to 5) that extend between the inner and outer surfaces 2a, 2b.

Finally, the cushion 2 has at least one front surface 2e (FIGS. 1 to 3) and at least one rear surface 2f (FIGS. 4 and 5) arranged, the first at the hand M (FIGS. 1 to 3) of the user U corresponding to the traumatized or operated shoulder ST, the second at the elbow G (FIGS. 1, 2, 4 and 5) of the user U corresponding to the traumatized or operated shoulder ST.

The brace 1 also comprises suitable constraining means for the engagement of the forearm A of the user U corresponding to the traumatized or operated shoulder ST to the cushion 2.

Advantageously, the constraining means comprise at least one arm support sling 3 (FIGS. 1 to 3) that extends from the cushion 2, preferably from the outer surface 2b thereof. The arm support sling 3 has at least one free portion 4 (FIGS. 1 to 3) provided with at least one hooking element 4a (FIG. 1), optionally of the Velcro® type.

It is possible for the portion 4 of the arm support sling 3 to be provided with a plurality of hooking elements 4a distributed so as to permit the correct engagement thereof to the cushion 2.

As can be seen in FIGS. 1 to 3, the free portion 4 of the arm support sling 3 can be engaged to the cushion 2 through the hooking element 4a to delimit along with it at least one compartment 5 for housing the forearm A of the user U corresponding to the traumatized or operated shoulder ST.

In detail, the arm support sling 3 has dimensions and shapes such as to allow the engagement of the hooking element 4a to the upper surface 2c of the cushion 2 so that the compartment 5 for housing the forearm A of the user U, relative to the traumatized or operated shoulder ST, is, at least in part, delimited between the arm support sling 3 and the outer surface 2b of the cushion 2.

The arm support sling 3 described above can also be replaced by one or more belts or bands capable to keeping the forearm A constrained to the cushion 2 according to the best position for the recovery of the traumatized or operated shoulder.

Advantageously, the brace 1 is provided with an auxiliary stabilising band 6 (FIGS. 1 to 5), optionally made of elastic material, for blocking the arm B of the user U relative to the traumatized or operated shoulder ST at the elbow G thereof.

In particular, the stabilising band 6 has a first end 6a (FIGS. 4 and 5) that engages, optionally by stitching (preferably by Velcro®, in order to be able to adjust the length and inclination of the band 6 itself), the cushion 2, preferably the rear surface 2f thereof, and a second end 6b (FIG. 1), opposite the first end 6a, provided with at least one hooking element 6c, optionally of the Velcro® type, for the engagement of the cushion 2.

Advantageously, the auxiliary stabilising band 6 is extensible astride of the arm B of the user U to at least partially wrap the humerus O thereof and block it against the cushion 2.

Again with reference to the attached figures, the brace comprises at least one abdominal band 7, optionally adjustable length-wise, suitable for enveloping and/or surrounding at least one abdominal and lumbar area ZA of the user U.

The abdominal band 7 is advantageously engaged to the cushion 2, preferably by stitching of a first end 7a thereof (FIGS. 4 and 5) to the rear surface 2f thereof, and it extends from the latter on the opposite side with respect to the arm support sling 3. On the opposite side to the first end 7a, the abdominal band 7 has a second end 7b (FIGS. 1 to 3), optionally provided with a snap blocking device 8a (FIGS. 1-3) able to interact with a corresponding blocking device 8b (FIGS. 1 to 3), localized on the cushion 2, preferably at the front surface 2e thereof.

Advantageously, the abdominal band 7 is adjustable length-wise to keep the cushion 2 resting against the body C of the user U.

Again with reference to the attached figures, the brace 1 comprises at least one support harness 9 engaged to the cushion 2 to support the latter according to a predetermined position and level.

As can be seen in the attached figures, the support harness 9 has at least one rest portion 10 suitable for engaging at least one shoulder of the user U to support the cushion 2 and the arm support sling 3.

Advantageously, the support harness 9 is configured so as to engage, preferably in contact, the traumatized or operated shoulder ST of the user U, in other words the shoulder corresponding to the forearm A that engages the arm support sling 3 and the cushion 2 of the brace 1.

The support harness 9 has, between the cushion 2 and the rest portion 10, at least one triangular structure (FIGS. 4 and 5) suitable for remaining against the body C of the user U at the scapular area AS (FIGS. 4 and 5) of the traumatized or operated shoulder ST thereof.

The support harness 9 also has, between the triangular structure 11 and the rest portion 10, at least one transverse stabilisation belt 12 intended to wrap around the body C of the user U and stabilize the rest portion 10, passing beneath the contralateral axilla cavity AC of the user U on the opposite side to the cushion 2.

Going into detail, as can be seen in FIGS. 4 and 5, the triangular structure 11 has a first vertex 13 arranged substantially at the cushion 2, a second vertex 14 arranged substantially at a rear end 10a of the rest portion 10 of the support harness 9 and a third vertex 15 arranged substantially at the sagittal plane X of the user U.

Each vertex 13, 14, 15 can have a variable width according to the configuration necessary to allow the adaptation of the brace 1 to the body shape of the user U.

Preferably, the triangular structure 11 is made so as to allow the variation of the width of each vertex 13, 14, 15. Of course, the variation of the width of one of the vertices 13, 14, 15 of the triangular structure 11 determines the variation of the width of the other angles 13, 14, 15.

Therefore, the width of the vertices 13, 14, 15 of the triangular structure 11 gives it a reticular configuration capable of providing a barrier to the movement and/or rotation of the scapula relative to the traumatized or operated shoulder ST of the user U.

In accordance with the solution illustrated in figures and 5, the triangular structure 11 of the support harness 9 is provided with at least two belts 16, 17, each of which has a first end 16a, 17a engaged to the cushion 2, preferably to the rear surface 2f of the latter by means of corresponding stitching, and a second end 16b, 17b engaged to a central return element 18, suitably configured to remain in proximity or at the sagittal plane X of the user U.

The first belt 16 of the triangular structure 11, optionally adjustable length-wise, advantageously extends between the cushion 2 and the central return element 18 (FIGS. 4 and 5), preferably through just an upper return element 19, localized on the rear end 10a of the rest portion 10 of the support harness 9.

The second belt 17 of the triangular structure 11 also extends between the cushion 2 and the central return element 18 without however engaging any intermediate return element.

As can be seen in FIGS. 4 and 5, the first ends 16a, 17a of the first and second belt 16, 17 define the first vertex 13 of the triangular structure 11, the second ends 16b, 17b of the first and second belt 16, 17 define the third vertex 15 of the triangular structure 11, whereas the first belt 16 defines the second vertex 14 of the triangular structure 11 at the upper return element 19.

Advantageously, the central return element 18 has an annular structure, optionally circular, for the engagement of at least one of the belts 13, 14, 15 of the triangular structure 11 of the support harness 9.

The annular structure of the central return element 18 lies on a plane substantially parallel to the body C of the user U during the use of the brace 1 avoiding unpleasant and painful sensations typical of solid bodies sticking into the skin.

According to a further advantageous aspect of the present invention, the upper return element 19 comprises at least one hinging point 20 (FIGS. 4 and 5) to the rear end 10a of the rest portion 10 of the harness 9. In this way, the upper return element 19 is free to rotate, with respect to the rest portion 10, about the hinging point 20 to allow the variations in width of the second vertex 14 of the triangular structure 11.

The upper return element 19 also has at least one slit 20a suitable for receiving—passing through it—a respective belt, preferably the first belt 16, of the triangular structure 11.

As an alternative to the embodiment shown in FIGS. 4 and 5, the triangular structure 11 can also be made through a first belt that extends just between the cushion 2 and the rear end 10a of the rest portion 10 and a second belt that extends between the cushion and the second end 10a of the rest portion 10 through the central return element 18. In this case, the upper return element 19 can be provided with a two-loop structure oriented so as to receive in engagement the corresponding ends of the belts of the triangular structure 11 or two independent bodies, one dedicated to each belt. Unlike the embodiment represented, the second vertex 14 of the triangular structure 11 would, in this case, be defined by the first and by the second belt, whereas the third vertex 15 would be defined just by the second belt at the central return element 18.

It is also possible for the triangular structure 11 to be able to be made through the use of three belts: a first belt extending between the cushion 2 and the rear end 10a of the rest portion 10; a second belt extending between the rear end 10a of the rest portion 10 and the central return element 18; and a third belt extending between the cushion 2 and the central return element 18. Also in this case it is possible to use an upper return element structured so as to have two loops each dedicated to a respective belt or an upper return element that is provided with two independent bodies, each dedicated to a respective belt.

Finally, it is possible for the triangular structure 11 to be made through a single belt having a first end fixed to the cushion 2 and a second end able to be engaged so as to allow length-wise adjustment. In this case, the belt defines, at the return elements 18 and 19, the third and the second vertex 15, 14 of the triangular structure 11 that would be structured so as to allow the belt itself to be crossed and folded over.

The aforementioned transverse stabilisation belt 12 of the support harness 9 comprises a first end 12a (FIGS. 1 to 3) engaged to a first front return element 21, which rotatably engages a front end 10b of the rest portion 10 of the support harness itself, and a second end 12b engaged to the central return element 18.

Alongside the first front return element 21 of the support harness 9 is further provided with a second front return element 22, also rotatably engaged to the front end 10b of the rest portion 10. The second front return element 22 supports a front support belt 23, preferably adjustable length-wise, which engages, on the opposite side with respect to the second front return element 22, the cushion 2, in particular the front surface 2e thereof advantageously by means of the corresponding stitching.

As can be seen in FIGS. 1 to 3, the support belt 23 is arranged vertically, in other words substantially parallel to the sagittal plane (X) of the user U, between the rest portion 10 and the cushion 2 so that just the traumatized or operated shoulder ST of the user U can be engaged by the rest portion 10 of the harness 9.

As can be seen in the attached figures, the adjustment of the belts 12, 16, 17, 23 and of the abdominal band 7 is preferably obtainable through the provision of corresponding hooking elements 24, optionally of the Velcro® type, at the free ends thereof. Such hooking elements 24 allow the ends folded around the corresponding return elements 18, 19, 21, 22 along the extension of the corresponding belts 12, 16, 17, 23 to be blocked.

The brace described above solves the problems encountered in the prior art and achieves important advantages.

Firstly, the brace described above allows the optimal immobilization of the traumatised shoulder even containing the movements of the corresponding scapula.

The arrangement of the rest portion at the traumatized or operated shoulder allows an increase in comfort of the brace that is consequently reflected in the rehabilitation power thereof, since the user is no longer induced to take up incorrect positions or postures, being able to use the brace for particularly long time periods.

In fact, the elimination of the contralateral rest portion avoids a transversal pressure on the neck of the user that was the cause of soreness and pain. The rest portion localized directly on the traumatized or operated shoulder is connected substantially vertically to the cushion and to the arm support sling, so that the load relative to the arm of the user only bears down on the shoulder thereof, avoiding diagonally involving the base of the neck that is very sensitive to stresses.

Keeping a correct rehabilitation posture and/or position over time allows a considerable reduction in the overall recovery time of the traumatized or operated shoulder also avoiding involving parts of the body not directly affected by the trauma or by the surgical operation.

1-9. (canceled)

10. A brace for immobilising a traumatized or operated shoulder, or for shoulders subjected to repair of rotator cuffs, subluxations, dislocations, Bankart procedures, repair of soft tissues following traumas, external rotation immobilisation, plastic capsular repair procedures or anterior/posterior stabilisations procedures, the brace comprising:
at least one cushion configured to at least partly adhere to a body of a user having a traumatized or operated shoulder to be immobilised;
constraining means for engagement of a forearm of the user, corresponding to the traumatized or operated shoulder, to the cushion;
at least one abdominal band, configured to envelope and/or surround at least one abdominal and lumbar area of the user, the abdominal band being engaged to the cushion and extending from the cushion on an opposite side with respect to the constraining means, the abdominal band configured to maintain the cushion against the body of the user;
at least one support harness engaged to the cushion for supporting the cushion according to a predetermined position and level, the support harness including at least one rest portion configured to engage at least one shoulder of the user for supporting the cushion and the constraining means,
wherein the support harness comprises at least one front support belt arranged vertically or substantially parallel to a sagittal plane of the user, between the rest portion and the cushion to engage, or rest thereon, the shoulder corresponding to the forearm which engages the constraining means and cushion of the brace.

11. A brace according to claim 10, wherein the support harness includes:
at least one triangular structure configured to remain against the body of the user at the scapular area of the traumatized or operated shoulder of the user, the triangular structure being localized between the cushion and the rest portion of the support harness;
at least one transverse stabilization belt for surrounding the body of the user on the side opposite to the cushion, the stabilization belt being localized between the triangular structure and the rest portion of the support harness.

12. A brace according to claim 11, wherein the triangular structure includes:
a first vertex arranged substantially at the cushion;
a second vertex arranged substantially at a rear end of the rest portion of the support harness;
a third vertex arranged substantially at the sagittal plane of the user, each vertex having a variable width, to define a containment net capable of providing a barrier against movement and/or rotation of the scapula of the user with respect to the traumatized shoulder.

13. A brace according to claim 11, wherein the triangular structure includes:
a first belt including a first end engaged to the cushion and a second end, opposite to the first end, engaged to a central return element configured to remain in proximity or at the sagittal plane of the user, the first belt extending between the cushion through at least one upper return element, localized on the rest portion of the support harness,
a second belt including a first end engaged to the cushion and a second end, opposite to the first end, engaged to the central return element, the second belt extending between the cushion and the central return element without crossing the intermediate return elements;
the first ends of the first and second belt defining the first vertex of the triangular structure, the second ends of the first and second belt defining the third vertex of the triangular structure, the first belt defining, at the upper return element, the second vertex of the triangular structure.

14. A brace according to claim 13, wherein the upper return element includes:
at least one point for hinging to the rest portion of the support harness, the upper return element being rotatable with respect to the rest portion at the hinging point;
at least one slit configured to receive, crossing, a respective belt of the triangular structure.

15. A brace according to claim 13, wherein the central return element has an annular or circular structure for engagement of at least one belt, the structure of the central return element lying on a plane substantially parallel to the body of the user during use of the brace.

16. A brace according to claim 13, wherein the stabilization transversal belt of the support harness includes:
a first end engaged to a first front return element rotatably engaged to a front end of the rest portion of the support harness;
a second end engaged to the central return element on the side opposite to the triangular structure of the support harness.

17. A brace according to claim 10, further comprising at least one auxiliary stabilizing band for blocking an arm of the user with respect to the traumatized or operated shoulder at a relative elbow thereof, the stabilizing band having a first end engaged to the cushion and a second end, opposite to the first end, including at least one hooking element for engagement thereof to the cushion.

18. A brace according to claim 10, wherein the means for constraining the forearm of the user corresponding to the traumatized or operated shoulder comprises at least one arm support sling, the arm support sling extending from the cushion for delimiting, with the cushion, at least one compartment for housing the forearm.
